SEQUENCE LISTING (4 sequences)

SEQ ID NO: 1 - protein, 9 aa, Unknown
  human
CDPGYIGSR

SEQ ID NO: 2 - protein, 19 aa, Unknown
  human
CSRARKQAASIKVAVSADR

SEQ ID NO: 3 - protein, 4 aa, Unknown
  human
GPRP

SEQ ID NO: 4 - protein, 4 aa, Unknown
  human
GPRP

(19) United States
(12) Patent Application Publication
(10) Pub. No.: US 2009/0274737 A1
(43) Pub. Date: Nov. 5, 2009

(54) IMPLANT COMPRISING A SURFACE OF REDUCED THROMBOGENICITY

(75) Inventor: Alexander Borck, Aurachtal (DE)

Correspondence Address: BRYAN CAVE POWELL GOLDSTEIN, ONE ATLANTIC CENTER FOURTEENTH FLOOR, 1201 WEST PEACHTREE STREET NW, ATLANTA, GA, 30309-3488, US

(73) Assignee: BIOTRONIK VI PATENT AG, Baar (CH)

(21) Appl. No.: 12/435,192

(22) Filed: May 4, 2009

(30) Foreign Application Priority Data
May 2, 2008 (DE) 10 2008 021 894.4

(51) Int. Cl. A61K 9/00; A61K 38/02; A61K 31/715; A61K 31/74; A61K 38/07; A61K 31/352; A61P 7/02; A61F 2/06

(52) U.S. Cl. 424/422; 514/2; 514/54; 424/78.08; 514/18; 514/457; 623/1.15; 977/754

(57) ABSTRACT

An implant for a human or animal body, comprising a surface having reduced thrombogenic properties, whose surface has a wetting angle of Θ, where Θ≦80°. Also disclosed is a method for producing an implant and the use an implant to reduce the dose or concentration in administration of a concomitant systemic medication with one or more anticoagulant active ingredients before, during or after implantation of the implant in a human or animal body.

### PRIORITY CLAIM

This patent application claims priority to German Patent Application No. 10 2008 021 894.4, filed May 2, 2008, the disclosure of which is incorporated herein by reference in its entirety.

### FIELD

The present disclosure relates to implants for a human or animal body, comprising a surface having reduced thrombogenic properties, a method for manufacturing implants and use of implants to reduce the dose and/or concentration in administration of concomitant systemic medication with one or more anticoagulant active ingredients before, during and/or after use of the implant in a human or animal body.

### BACKGROUND

Implants are substance or parts introduced into the human or animal body to fulfill certain substitute functions for a limited period of time or for life. In contrast with transplants, implants consist of artificial material (also referred to as alloplasty). A distinction is often made between medicinal implants, plastic implants and functional implants.

Medicinal implants have the function of supporting or replacing body functions or structures. Depending on the function, medicinal implants are also referred to as implantable prostheses. Known representatives include, for example, cardiac pacemakers, cerebral pacemakers for Parkinson's disease, cardiac implants, cochlear implants, dental implants, stents and implants that serve to form a depot of a pharmaceutical substance as well as various forms of joint replacement.

Plastic implants are used in plastic surgery, e.g., to replace destroyed body parts or to alter existing body parts.

Functional implants serve to monitor human or animal functions, e.g., by subcutaneous implantation of radiofrequency identification (as referred to as RFID) chips.

On the basis of the variety of types of implants available, it can be seen that implants and their use have acquired a great significance in medicine.

With traditional treatment principles, as in systemic administration of one or more active ingredients, for example, substantial adverse effects are to be expected in some cases, e.g., in oncotherapy, so that local, controlled release of the active ingredients at or in proximity to the target site is becoming increasing important (also referred to as local drug delivery or “LLD” concept). To be able to perform this local administration of active ingredients, implant base bodies, in particular, are coated with active ingredients which are implanted either at or in proximity to the target site in a human or animal body and thus release active agents locally. This clinically established method is used millions of times each year throughout the world, and it is to be expected that the demand for new materials and new forms of administration will increase taking into account the demographic shift within the age pyramid.

In the orthopedic field, implant-associated infections and thromboembolic complications are known in conjunction with endoprosthetic implants. A thromboembolism is an acute venous or arterial vascular occlusion occurring due to a thrombus carried in the blood stream, which may occur due to platelets adhering to the surface of the implant. Emboli, in particular, pulmonary emboli, are the most common forms of thromboembolism.

In the field of cardiovascular diseases, minimally invasive forms of treatment for dilating and stabilizing stenosed coronary vessels through percutaneous transluminal coronary angioplasty (also referred to as PTCA) and stent implantation are an increasingly popular treatment method. In addition to reocclusion of the vessel after stent implantation (in-stent restenosis, also referred to as ISR) and tissue inflammation, the main late complication to be mentioned here is the risk of thrombosis.

On the basis of these examples, the importance of reducing the risk of thrombosis and/or thromboembolism after implantation of the implant becomes clear. To achieve this, a concomitant medication in the form of one or more anticoagulants is currently being administered systemically to the human or animal receiving the implant. The gold standard, i.e., the concomitant medication of choice, has proven to be “dual anti-platelet therapy” in which aspirin and clopidogrel, for example, are administered systemically as anticoagulants. Such a concomitant medication is usually administered systemically as long as the implant in the human or animal body causes platelets or other components of blood to adhere to the surface of the implant. This usually means that the concomitant medication must be continued for months or years or even until death of the person or animal to reduce the risk of thrombosis/embolism.

Some substantial adverse effects are to be expected due to the active-ingredient properties of anticoagulant substances, in particular, aspirin and clopidogrel.

Primarily CNS disorders are to be observed with chronic overdoses of aspirin, also known as “salicylism,” whereas mainly the acid-base equilibrium in the animal or human body is disturbed in an acute overdose, sometimes to a substantial extent, and initial central hyperventilation can develop into a respiratory alkalosis. A renal compensation attempt with alkaluria may lead to loss of potassium and chloride as well as water (the loss of water is due to vomiting). A wide variety of syndromes may be observed, e.g., tinnitus, nausea, vomiting, impaired vision and hearing, headaches, dizziness and confusion.

With clopidogrel, bleeding/hemorrhages are observed, in particular, as an adverse effect; gastrointestinal bleeding and other bleeding, such as purpura, bruises, hematomas and nosebleeds, in particular, are often observed. Hematomas, hematuria and ocular hemorrhages are observed less often and intracranial hemorrhages are observed occasionally.

With the combination of aspirin and clopidogrel, a significantly increased risk for mild, severe and other bleeding, primarily in the gastrointestinal area, or bleeding in the area of puncture sites is observed. It has been found that the incidence of severe bleeding is a function of the aspirin dose and declines in the course of treatment (CURE study).

If a patient with an implant requires an additional medical procedure, especially dental procedures or other surgical procedures in the field of cardiology or knee and hip replacements, in particular, then concomitant systemic medication with anticoagulants should be interrupted to avoid increasing the incidence of hemorrhage during and after the respective procedure. However, this results in an increased risk of thrombosis/embolism due to the implant.

The present invention reduces the risks attributed to the implant itself, in particular, the risk of thrombosis/embolism, while reducing the adverse effects, in particular, bleeding, caused by the concomitant medication.

### SUMMARY

The present disclosure describes several exemplary embodiments of the present invention.

One aspect of the present disclosure provides an implant for a human or animal body, wherein the surface of the implant has a wetting angle of Θ, where Θ≦80°.

Another aspect of the present disclosure provides a method for producing an implant, comprising a) providing an implant base body; and b) treating the implant base body such that the surface of the implant has a wetting angle of Θ, where Θ≦80°.

A further aspect of the present disclosure provides a method of reducing the dose and/or duration of administration of a concomitant systemic medication with one or more anticoagulant active ingredients, before, during and/or after implantation in a human or animal body, comprising implanting an implant for a human or animal body, wherein the surface of the implant has a wetting angle of Θ, where Θ≦80°.

An additional aspect of the present disclosure provides a method for reducing the dose or duration of administration of a concomitant systemic medication with at least one anticoagulant active ingredient, before, during or after implantation of an implant in a human or animal body, comprising implanting in a human or animal body an implant whose surface has a wetting angle of Θ, where Θ≦80°.

Exemplary embodiments of the present invention are described in the detailed description hereinbelow and can be combined with one another, if appropriate.

### BRIEF DESCRIPTION OF THE DRAWINGS

Various aspects of the present disclosure are described hereinbelow with reference to the accompanying figures. The figures show a schematic detail of hyperbranched polymers, in particular, star polymers, to be used as anticoagulants for inventive implants.

FIG. 1 shows a schematic detail of a hyperbranched polymer structure; and

FIG. 2 shows a schematic detail of a star polymer structure.

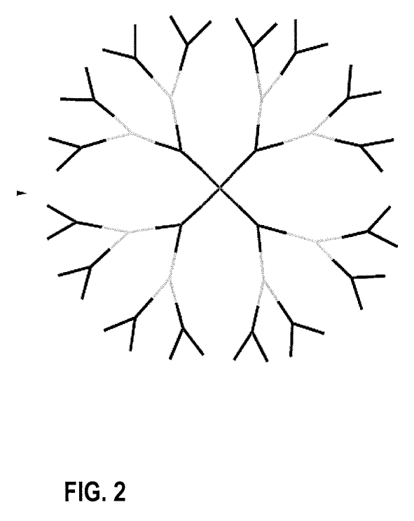

### DETAILED DESCRIPTION

The implants of the present disclosure address the present problem because the surface of the inventive implants has a wetting angle Θ, where Θ≦80° which provides improved and, in particular, accelerated endothelialization of the implants.

Based on the improved and accelerated endothelialization of the implant of the present disclosure, endothelial cell growth over the surface of the implant is accelerated and thus the adhesion of platelets and/or other components of blood that can cause a thrombosis, i.e., a thromboembolism, is reduced or even prevented. Consequently, the risk of thrombosis/embolism after implantation of the implant is reduced; and, therefore, the dose and/or concentration in administration as well as the duration of a concomitant systemic medication with one or more anticoagulants can also be reduced.

For purposes of the present disclosure, the meaning of a wetting angle Θ, where Θ≦80° for the surface of the implant is defined hereinbelow.

After applying a drop of water under standard conditions according to the sessile drop method, the wetting behavior of the drop as a function of the surface energy of the substrate is such that it is manifested in a wetting angle of Θ≦80°. As an alternative to the experimental method, the wetting angle may be calculated by conventional methods. To do so, the Du Noüy ring method or the Wilhelmy plate method, in particular, may be used. In these methods, the angle can be calculated with a known surface tension of the fluid.

For purposes of the present disclosure, the phrase “treatment of the surface of an implant base body so that the surface has a wetting angle of Θ≦80°” means that the surface of the implant may usually be triggered to hydrophilize the surface and thus to establish a wetting angle Θ≦80° by selection of (i) suitable implant materials and/or (ii) suitable surface modifications by means of suitable hydrophilic substances.

For purposes of the present disclosure, implants and/or implant base bodies may include any medical, plastic and/or functional implants and/or implant base bodies and are selected, for example, from the group consisting of cardiac pacemakers; cerebral pacemakers and defibrillators; cardiac implants, in particular, heart valves, but not limited thereto; pacemaker electrodes; defibrillation electrodes; cochlear implants; penile implants; dental implants; endoprostheses, preferably for knee and hip joints; depot implants that serve to form a depot of an active ingredient; biodegradable or permanent coronary or peripheral stents; biodegradable or permanent stents for other cavities, preferably the esophagus, the bile ducts, the urethra, the prostate or the trachea; and local drug delivery (LDD) implants, which are preferably implanted endovascularly in the blood stream or other cavities.

In one exemplary embodiment of the present disclosure, implants are selected from the group consisting of cardiac pacemakers; defibrillators; cardiac implants, preferably heart valves; pacemaker electrodes; defibrillation electrodes; biodegradable or permanent coronary or peripheral stents; and local drug delivery (LDD) implants, which are preferably implanted endovascularly in the blood stream or other cavities.

In another exemplary embodiment of the present disclosure, implants are selected from the group consisting of permanent or biodegradable coronary stents (e.g., coronary stents), where the stent base body material may include metals and/or polymers.

The original mechanical functions of a coronary stent, e.g., its dilatability, low recoil, stability over a desired period of time (in the case of degradable stents, e.g., comprising magnesium and alloys thereof) as well as flexibility, are preferably present in stents as implants.

Implant materials to be used according to the present disclosure, preferably stent base body materials and exemplary embodiments thereof, are described hereinbelow.

### Biodegradable Implant Base Bodies, in Particular Biodegradable Stent Base Bodies

For purposes of the present disclosure, the term “biodegradable implant (base body),” in particular, “biodegradable stent (base body),” means that the base body is degraded in a physiological environment, in particular, in the vascular system of a human or animal body, so that the stent loses its integrity. Biodegradable implant base bodies preferably degrade only when the function of the implant is no longer physiologically appropriate and/or necessary. In the case of biodegradable stents, this means that the stent preferably degrades or loses its integrity only when the traumatized tissue of the vessel has healed and the stent need no longer exert its supporting function in the vessel.

### Metallic Base Bodies

In one exemplary embodiment, the biodegradable material preferably comprises a metallic material, which is a biocorrodable alloy, the main components of the alloy being selected from the group consisting of magnesium, iron, zinc and tungsten. A magnesium alloy is preferred for a degradable metallic material.

The composition of the alloy comprising, in particular, magnesium, iron, zinc and/or tungsten is to be selected to be biocorrodable. For purposes of the present disclosure, the term “biocorrodable” refers to alloys in which degradation takes place in a physiological environment, ultimately leading to the entire stent or the part of the stent formed from this material losing its mechanical integrity. For purposes of the present disclosure, the term “alloy” means a metallic structure whose main component is magnesium, iron, zinc or tungsten. The main component is the alloy component present in the alloy in the largest amount by weight. The amount of the main component is preferably more than 50 wt %, more preferably more than 70 wt %. A magnesium alloy is preferred.

If the material is a magnesium alloy, it preferably contains yttrium and other rare earth metals, because such an alloy is characterized by its physicochemical properties and its high biocompatibility, in particular, its degradation products.

Magnesium alloys of the WE series, in particular, WE43, as well as magnesium alloys of the following composition are especially preferred: rare earth metals 0.05-9.9 wt % including yttrium 0.0-6.5 wt % and the remainder <1 wt %, which may include zirconium and/or silicon, with magnesium accounting for the rest of the alloy to a total of 100 wt %. These magnesium alloys have already confirmed their special suitability in experimental studies and preliminary clinical trials, i.e., the magnesium alloys have a high biocompatibility, favorable processing properties, good mechanical characteristics and satisfactory corrosion behavior for the use purposes. For purposes of the present disclosure, the umbrella term “rare earth metals” includes scandium (21), yttrium (39), lanthanum (57) and the 14 elements following lanthanum (57), namely, cerium (58), neodymium (60), promethium (61), samarium (62), europium (63), gadolinium (64), terbium (65), dysprosium (66), holmium (67), erbium (68), thulium (69), ytterbium (70) and lutetium (71).

### Polymer Base Bodies:

According to another exemplary embodiment, implant base bodies, in particular, stent base bodies, may comprise biodegradable polymers, preferably selected from the group consisting of polydioxanone; polyglycolide; polycaprolactone; polyhydroxyvaleric acid; polyhydroxybutyric acid; polylactides, preferably poly(L-lactide), poly(D-lactide), poly(D,L-lactide) and blends as well as copolymers, and preferably poly(L-lactide-co-glycolide), poly(D,L-lactide-co-glycolide), poly(L-lactide-co-D,L-lactide), poly(L-lactide-co-trimethylene carbonate), poly-ε-caprolactone, poly(L-lactide-co-ε-caprolactone and triblock copolymers; polyester amide; polysaccharides, preferably chitosan, alginate, carrageenan, levan, hyaluronic acid, heparin, dextran and cellulose or cellulose derivates, such as nitrocellulose and polypeptides.

### Permanent Implant Base Body, Preferably Permanent Stent Base Body:

In contrast with the biodegradable base body, the “permanent implant base body,” preferably the “permanent stent base body,” essentially does not degrade in a physiological environment in the human or animal body, so the permanent implant base body retains its integrity.

### Metallic Base Bodies:

In another exemplary embodiment, the base body comprises a permanent implant, in particular, a permanent stent, preferably from a shape memory material selected from one or more materials from the group consisting of nickel-titanium alloys and copper-zinc-aluminum alloys, preferably nitinol.

In yet another exemplary embodiment, the base body of a permanent implant, in particular, a permanent stent, comprises stainless steel, preferably a Cr—Ni—Fe steel, here especially the alloy 316L, or a Co—Cr steel.

### Polymer Base Body

In an additional exemplary embodiment, the base body of a permanent implant, in particular, a permanent stent, preferably comprises polypropylene, polyethylene, polyvinyl chloride, polymethylmethylethyl acrylate, polymethylethyl acrylate, polytetrafluoroethylene, polyvinyl alcohol, polyurethane, polybutylene terephthalate, silicone, polyphosphazene as well as their copolymers and blends or polyhydroxybutyric acid (atactic, isotactic, syndiotactic and blends thereof).

The present invention also provides permanent implants, preferably stents, in particular, made of metal, or biodegradable implants, preferably stents, made of polymer, because these implants remain in the body permanently or for a long period of time and, therefore, the risk of thrombosis/embolism is high per se.

In contrast, metallic biodegradable implants, preferably magnesium stents, degrade comparatively rapidly, so that sometimes the implant can no longer exercise its supporting functionality over the desired period of time. However, diffusion of liquid, in particular, water to the implant material is reduced because of the comparatively rapid endothelialization of a biodegradable metallic implant, preferably a stent. The degradation can thus be delayed to the extent that the implant can exert its supporting functionality over the entire desired period of time, while at the same time reducing the risk of thrombosis/embolism.

In a further exemplary embodiment, the base body of the implant, preferably a stent, may additionally comprise plastics, preferably polyurethane and/or ceramics and/or other polymer coatings.

If endovascularly implantable stents are used as the implantable base bodies, all the conventional stent geometries may be used. Especially preferred are the stent geometries described, in particular, in U.S. Pat. No. 6,896,695; U.S. Patent Application No. 2006/241742; U.S. Pat. No. 5,968,083 (Tenax); European Patent Application No. 1 430 854 (helix design); U.S. Pat. No. 6,197,047; and European Patent Application No. 0 884 985.

According to another exemplary embodiment, in order for the surface of the inventive implants, preferably stents, to have a wetting angle of Θ≦80°, the implant and/or stent base body material may be selected from the groups consisting of:
permanent metallic materials: 316L, nitinol and Co—Cr, whereby the materials may be used alone or in combination with a coating of silicon carbide (coated according to the CVD method) as the implant base body, preferably the stent base body;
permanent polymer materials: polypropylene, polyethylene, polyvinyl chloride, polymethylmethylethyl acrylate, polymethylethyl acrylate, polytetrafluoroethylene, polyvinyl alcohol, polyurethane, polybutylene terephthalate, silicone, polyphosphazene as well as their copolymers and blends or polyhydroxybutyric acid (atactic, isotactic, syndiotactic and blends thereof);
biodegradable metallic materials: magnesium alloys, especially preferably magnesium alloys as described hereinabove; and
biodegradable polymer materials: polydioxanone; polyglycolide; polycaprolactone; polyhydroxyvaleric acid; polyhydroxybutyric acid; polylactides, preferably poly(L-lactide), poly(D-lactide), poly(D,L-lactide) and blends as well as copolymers, and preferably poly(L-lactide-co-glycolide), poly(D,L-lactide-co-glycolide), poly(L-lactide-co-D,L-lactide), poly(L-lactide-co-trimethylene carbonate), poly-ε-caprolactone, poly(L-lactide-co-ε-caprolactone and triblock copolymers; polyesteramide; polysaccharides, preferably chitosan, alginate, carrageenan, levan, hyaluronic acid, heparin, dextran and cellulose or cellulose derivatives such as nitrocellulose and polypeptides.

Alternatively or in addition to the methods described hereinabove, the surface of an implant and/or stent base body may be modified with (a) one or more hydrophilic substances, which may be the same or different, so that the surface of the implant has a wetting angle of Θ≦80°. For purposes of the present disclosure, “modified” means that the surface of the implant, preferably a stent, is coated so that one or more hydrophilic substances, which may be the same or different, adhere permanently to the surface of the implant and/or stent and are not released to the body after implantation. The usual coupling methods are described in Examples 1 to 3 or the coupling methods are explained in the following literature citation: G. T. Hermanson; Bioconjugate Techniques: 1996, Academic Press, ISBN 0-12-342336-8.

In one exemplary embodiment the (a) hydrophilic substances are selected from the group consisting of hyaluronic acid, preferably crosslinked or derivatized hyaluronic acid; chondroitin sulfate; extracellular matrix polypeptides or oligopeptides of SEQ ID No. 1 or SEQ ID No. 2 and fragments or derivatives thereof.

In another exemplary embodiment, the surface of an implant, preferably a stent, is additionally coated with (b) one, two or more anticoagulants, which may be the same or different.

For purposes of the present disclosure, an active ingredient is a substance or a compound that induces a biological reaction in a human or animal body. An anticoagulant active ingredient, therefore, induces an anticoagulant response in the human or animal body. In this sense, the term “active ingredient” may also be synonymous with pharmaceutical substance and/or drug.

In another exemplary embodiment, (b) one, two or more anticoagulant ingredients, which are the same or different, are permanently bound to the surface of the implant and/or stent, so the anticoagulant ingredients need not be delivered to the body after implantation. One or more anticoagulant ingredients, in particular, peptides of SEQ ID No. 3 and SEQ ID No. 4, may also have hydrophilic properties and may additionally support the establishment of the wetting angle of Θ≦80° and thus support improved endothelialization and, in particular, accelerated endothelialization of the implants of the present disclosure. In addition, the endothelialization may be further supported by the fact that the adherence of platelets and/or other blood components, which could cause a thrombosis and/or embolism, to the surface of the implant is reduced or even prevented directly by the anticoagulant active ingredients. Consequently, the implants, preferably stents, which additionally have (b) one or more anticoagulant active ingredients, are preferred, because these implants contribute to a reduction in the dose and/or concentration on administration of a concomitant systemic medication with one or more anticoagulant active ingredients.

In yet another exemplary embodiment, the anticoagulant active ingredients are selected from the group consisting of anticoagulant peptides, preferably peptides of SEQ ID No. 3 or SEQ ID NO. 4 or fragments or derivatives thereof; glucosamine glycans, preferably heparin; vitamin K antagonists, preferably coumarin, dicoumarol, phenprocoumon, warfarin and acenocoumarol; sulfated anticoagulant polymers, preferably sulfated hyperbranched polymers; sulfated star polymers; and dendrimers, preferably sulfated dendrimers.

In an additional exemplary embodiment, (b) the anticoagulant active ingredients are selected from the group consisting of peptides of SEQ ID No. 3 or SEQ ID No. 4 or fragments or derivatives thereof; coumarin, phenprocoumon, warfarin and acenocoumarol; sulfated star polymers; sulfated hyperbranched polymers; dendrimers and sulfated dendrimers.

For purposes of the present disclosure, the term “hyperbranched polymers” includes all macromolecules having strong branching in a regular or irregular form.

For purposes of the present disclosure, the term “star polymer” means that the polymer forms a subunit of hyperbranched polymers in which three or more chains emanate from a center. The center may be a single atom (e.g., nitrogen) or an atomic group (e.g., an organic hydrocarbon compound, especially in ring form). Star polymers may either contain arms of the same length and composition or may have an asymmetrical structure, i.e., different arm lengths and block copolymer chains.

For purposes of the present disclosure, the term “dendrimer” denotes a special subunit of star polymers in which additional branching occurs in the arms.

Whereas dendrimers are constructed step by step, the “simpler” highly branched structures are synthesized in one approach by conversion of a monomer of the structure ABₙ having one reactive A group and n reactive B groups. Reaction of the A groups with the B groups forms randomly branched molecules. This does not result in crosslinking reactions because the B groups are present in excess and there are too few “partners” to form network structures.

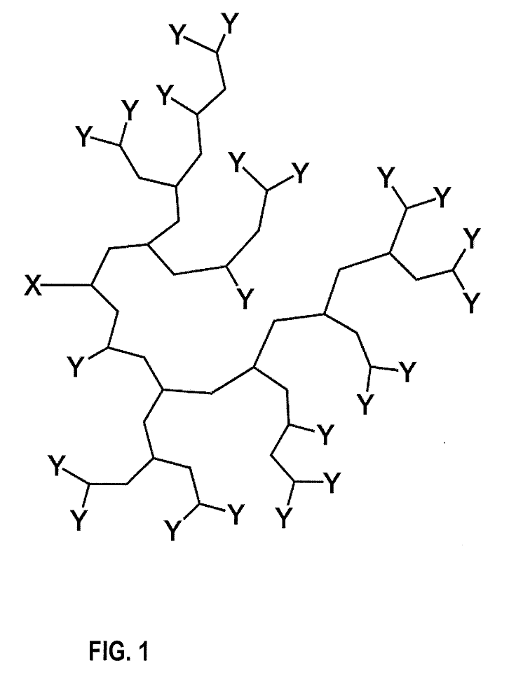

The following literature citations describe synthesis methods for hyperbranched polymers (see also FIG. 1; 4=SO₃), preferably star polymers: J. G. Zilliox, P. Rempp, J. Parrod: Pol. Sci. C, 22, 145, (1966) and P. Rempp, E. Franta: Pure and Appl. Chem., 30, 229, (1972). Synthesis of a star polymer from styrene and divinylbenzene is described as adding divinylsulfone first after the addition of styrene is terminated. This forms polymers with double bonds at the chain end with one-sided growth at the same time, and star polymers to be used according to the present disclosure are formed by reaction on the nongrowing chain.

An alternative method for synthesizing hyperbranched polymers, preferably star polymers, can be performed by means of anionic polymerization and is described in the following literature citations: M. Nagasawa, T. Fujimoto: Progr. Pol. Sci. Japan, 2, 263, (1972). A polyfunctional anion is used as the initiator here so that a macromolecule grows in a star pattern toward all sides. Polyfunctional initiators having multiple anionic radicals are obtained by polymerization of divinylbenzene with butyllithium in dilute solution (H. Eschwey, M. L. Hallensleben, W. Burchard: Makro. Ch., 173, 235-239, (1973)).

The following literature citation describes a sulfation method using an SO₃-pyridine complex for hyperbranched polymers, preferably star polymers and dendrimers (A. Sunder, R. Hanselmann, H. Frey, R. Müllhaupt: Macromolecules, 32, (1999); A. Sunder, R. Mühlhaupt, R. Haag, H. Frey: Macromolecules, 33, 253, (2000)).

Usually the one or more anticoagulant active ingredients (b), which may be the same or different, are bound to functionalized surfaces of implants. The surfaces may be dopaminized or silanized, for example (Example 3). Non-restrictive examples in this regard are presented in Examples 4 and 5.

Anticoagulant peptides may also be bound to the surface of the implants, preferably stents, by means of conventional coupling reactions, such as those also used for immobilization of enzymes. These include the methods of ionotropic gelation, e.g., by means of alginate or chitosan, and, in particular, simplex gelation, e.g., by means of alginate-chitosan. Suitable methods are described, in particular, in the dissertation by Alexander Borck, “Synthesis and Investigation of Biocompatible Materials for Medical Technical Applications,” University of Braunschweig; URL: http://www.digibib.tu-bs.de/?docid=00000014; chapter 2.1.1 with additional references there.

Sulfated polymers, preferably sulfated hyperbranched polymers, more preferably sulfated star polymers, as well as sulfated dendrimers, may usually be bound as monolayers to the surface of implants, preferably stents, by means of covalent bonds or by means of ionic interactions, in particular, ionotropic gelation with cationic polyelectrolytes, e.g., chitosan, polydiallyldimethylammonium chloride (poly-DADMAC) and polyethylene-imine in the form of simplex gels, e.g., alginate/chitosan (see in this regard the dissertation by Alexander Borck, “Synthesis and Investigation of Biocompatible Materials for Medical Technical Applications,” University of Braunschweig; URL: http://www.digibib.tu-bs.de/?docid=00000014; chapters 2.1.1.3; 3.2.1.1.4 and 4.1.2 with additional references there).

Alginate is usually converted to the water-insoluble state by polyvalent cations Ca²⁺ or Al³⁺, whereas, in the case of chitosan, a polyvalent phosphate is used. However, a simplex gel in which the polycation chitosan interacts with the polyanion alginate is also possible. The simplex gel is formed by metathesis, i.e., a double reaction.

Chitosan reacts with polyphosphate and leads to structurizing. Ca alginate reacts with the polyphosphate to form the poorly soluble Ca polyphosphate and soluble Na alginate which, in turn, interacts with the chitosan-bound polyphosphate forming alginate-chitosan, a simplex gel, which can then be used to form a monolayer coating on a stent surface. A non-restrictive example of this is presented in Example 4.

In another exemplary embodiment, the implant, preferably a stent, comprises a coating with an effective concentration of (c) one or more additional active ingredients, which may be the same or different, to treat late complications such as in-stent restenosis, tissue inflammation or other diseases, e.g., oncological diseases. For purposes of the present disclosure, the additional active ingredients (c) are not permanently bound to the implant, preferably a stent, but instead are released to the blood stream and/or the tissue of the human or animal body after implantation of the implant, preferably a stent.

The additional active ingredients (c) are, therefore, preferably selected from the group consisting of antiphlogistic drugs, preferably dexamethasone, methylprednisolone and diclofenac; cytostatics; taxols, preferably paclitaxel, colchicine, actinomycin D and methotrexate; immunosuppressants, preferably limus compounds, more preferably sirolimus (rapamycin) and derivatives thereof; zotarolimus (Abt-578); tacrolimus (FK-506); everolimus; biolimus, in particular, biolimus A9 and pimecrolimus; cyclosporin A and mycophenolic acid; platelet aggregation inhibitors, preferably abciximab and iloprost; statins, preferably simvastatin, mevastatin, atorvastatin, lovastatin, pitavastatin and fluvastatin; and estrogens, preferably 17β-estradiol, daizein and genistein; lipid regulators, preferably fibrates; immunosuppressants; vasodilators, preferably satanes; calcium channel blockers; calcineurin inhibitors, preferably tacrolimus; anti-inflammatories, preferably imidazoles; antiallergics; oligonucleotides, preferably decoy oligodeoxynucleotide (dODN); endothelium-forming agents, preferably fibrin; steroids; proteins/peptides; proliferation inhibitors; analgesics; and antirheumatics.

Paclitaxel and limus compounds are especially preferred according to the present disclosure, more preferably sirolimus (rapamycin), zotarolimus (Abt-578), tacrolimus (FK-506), everolimus, biolimus, in particular, biolimus A9 and pimecrolimus, most especially preferably rapamycin (sirolimus) as (c) the additional active ingredients.

A stent is preferably coated with the additional active ingredients (c) on the abluminal side, i.e., on the surface which is in contact with the tissue after implantation and is not in contact with the vascular lumen of the blood vessel because, with an additional luminal coating, the degradation of the stent, preferably a biodegradable stent and especially preferably a biodegradable metal stent, is significantly impaired.

In another exemplary embodiment, an implant coated with additional active ingredients may additionally have another coating (free of active ingredients) as a topcoat (d) to reduce the abrasion of the active ingredient coating in implantation.

The coating of the surface of the implant, preferably stent, with other active ingredients (c) is accomplished according to conventional methods. In particular, a pure active ingredient melt, an active ingredient solvent mixture or an active ingredient-polymer mixture may be applied to the surface of the implant by means of an immersion method (dip coating), a spray coating by means of single-component and/or multicomponent nozzle, rotary atomization and pressure nozzles, sputtering. The same coating methods may also be preferred for use with the topcoat (d).

For the case when one or more different polymers for the additional active ingredient coating (c) and/or the topcoat (d) are used, the polymers are generally selected from the group consisting of:
nondegradable polymers: polyethylene; polyvinyl chloride; polyacrylate, preferably polyethyl and polymethyl acrylate; polymethyl methacrylate; polymethyl-co-ethyl acrylate and ethylene/ethyl acrylate; polytetrafluoroethylene, preferably ethylene/chlorotrifluoroethylene copolymers; ethylene/tetrafluoroethylene copolymers; polyamides, preferably polyamideimide, PA-11, PA-12, PA-46, PA-66; polyether imide; polyether sulfone; poly(iso)butylene; polyvinyl chloride; polyvinyl fluoride; polyvinyl alcohol; polyurethane; polybutylene terephthalate; silicones; polyphosphazene; polymer foams, preferably polymer foams of carbonates; styrenes; copolymers and/or blends of the polymer classes listed; polymers of the class of thermoplastics; and
degradable polymers: polydioxanone; polyglycolide; polycaprolactone; polylactides, preferably poly-L-lactide, poly-D,L-lactide and copolymers as well as blends thereof, preferably poly(L-lactide-co-glycolide), poly(D,L-lactide-co-glycolide), poly(L-lactide-co-D,L-lactide), poly(L-lactide-co-trimethylene carbonate); triblock copolymers; polysaccharides, preferably chitosan, levan, hyaluronic acid, heparin, dextran, cellulose; polyhydroxyvalerate; ethylvinyl acetate; polyethylene oxide; polyphosphorylcholine; fibrin; albumin; polyhydroxybutyric acid, preferably atactic, isotactic and/or syndiotactic polyhydroxybutyric acids as well as blends thereof.

Especially preferred polymers for the active ingredient-containing layer (c) or the topcoat (d) of the present invention are the degradable polymers described hereinabove because no exogenous component remains in the body due to the complete degradation of the polymers.

For the case when primarily only the abluminal surface of a stent is to be coated with one or more other active ingredients (c), this may preferably be accomplished by mounting the stent on a cylinder, cannula or mandrel, for example, in the methods described hereinabove, so that only the abluminal surface of the stent is coated with a active ingredient layer. Alternatively, the abluminal coating may be performed with additional active ingredients by means of roller application or selected application by painting or filling cavities. The same methods may also preferably be used for the topcoat (d).

If necessary, a conventional drying step or other conventional physical or chemical post-processing steps, e.g., vacuum or plasma treatment, may follow one or more coating steps before the implant, preferably a stent, is treated further.

The exemplary embodiments of the implant usable according to the present disclosure, preferably a stent, may be combined with one another in all conceivable variants but also with the other preferred embodiments disclosed herein.

### EXAMPLES

The PRO-Kinetic stent, a cobalt-chromium stent with a ProBio coating consisting of a silicon carbide layer, is used as the stent base body.

The present invention is described by the following exemplary embodiments, although the exemplary embodiments do not limit the scope of protection of the present invention.

### Example 1

### Coupling to Carbonyldiimidazole (CDI)

A stent cleaned in an oxygen plasma or by rinsing with the solvent series of dichloromethane, acetone, methanol and Millipore water is treated further as described below.

A 1 mM solution of hydroxyundecylphosphonic acid in dry tetrahydrofuran is prepared. The stent is suspended in this solution and the solvent is evaporated within one hour, whereupon the meniscus of the solution travels over the stent surface.

The stent is then heated for 18 hours at 120° C. and next rinsed with solvent.

The stent pretreated in this way is placed in a 0.3M solution of carbonyldiimidazole (CDI) in dry dioxane for 15 hours. Next the stent is rinsed twice for 10 minutes with dry dioxane and then dried in a stream of nitrogen.

A solution of reagents to be coupled, such as the peptides described hereinabove (approximately 50 μg/mL) in PBS buffer (free of amino acid), is applied to the stents treated in this way and then shaken overnight at 4° C. Next the stents are rinsed with buffer.

### Example 2

### Coupling to 3-(4-oxybenzophenone)propylphosphonic Acid

A stent cleaned according to Example 1 is treated further as follows:

A 3 mM solution of 3-(4-oxybenzophenone)propylphosphonic acid in dry tetrahydrofuran is prepared.

The cleaned stent is sprayed three times with this solution. The stent is then heated for 12 hours at 120° C. and next rinsed with solvent.

These stents are placed in a solution of reagents to be coupled, such as the peptides described hereinabove (approximately 500 μg/mL), in buffer and shaken overnight at 4° C.

The stents are removed from the solvent the next day, then dried and exposed to 100 mW/cm² at 260 nm.

Unbound protein is washed off.

### Example 3

### Coupling with Silane

Batch:

The cleaned stents according to Example 1 are placed in a mixture of toluene, triethylamine and 3-aminopropyltriethoxysilane and incubated for 14 hours at room temperature. After the reaction is finished, the stent is washed in toluene and heated for one hour at 135° C.

Preparing the silanizing solution:

10 mL toluene, dried
0.5 mL triethylamine
1 mL silane (3-aminopropyltriethoxysilane)

Activation with 1,1′-carbonyldiimidazole (CDI) is performed following the cleaning step (rinsing the stents with trichloromethane). The quality of the CDI is crucial for success here.

The silanized and rinsed stents are placed in CDI for 5 hours, using the CDI dissolved in drying dioxane. A stock solution of 2.5 g/50 mL CDI in dioxane which is stable for several days (2, dry) is suitable for this. The stents are moved slightly at room temperature.

After the activation, the stents are removed and rinsed with drying dioxane.

For coupling of the peptides, the activated stents are immersed in the peptide solution and coupled at 4° C. overnight (at least 12 hours).

The reaction preferably takes place in 125 mM sodium borate with 0.066% SDS at a pH of 10.0.

The solution can then be reused and several surfaces can be treated with this solution

After coupling, the stents are washed three times with 5 mL of borax puffer (above), then three times with water. The peptides analyzable after these washing steps are covalently bonded.

### Example 4

### Binding of Anticoagulant Active Ingredients to dopaminized Implant Surfaces on the Example of Stent Surfaces

### 4.1 Dopaminizing a Stent Surface

The stent surface is brought in contact with a 1-3% L-dopamine solution in a 50 mM phosphate buffer solution (without the addition of NaCl) for 2-6 hours at 20° C.

### 4.2 Binding of an Anticoagulant Active Ingredient to the Dopaminized Stent Surface

The dopaminized stents are placed in CDI for 5 hours after dissolving the CDI in drying dioxane. A stock solution of 2.5 g/50 mL CDI in dioxane is suitable for this. The stents are moved slightly at room temperature.

After activation, the stents are removed and rinsed with drying dioxane.

For coupling the anticoagulant peptides or the polymers described here, the activated stents are immersed in the corresponding solution and coupled overnight (at least 12 hours) at 4° C.

The reaction most preferably takes place in 125 mM sodium borate with 0.066% SDS at a pH of 10.0.

The solution is then reusable, i.e., multiple surfaces can be treated with this solution.

The stents are washed three times with 5 mL of borax buffer (see hereinabove) after coupling, then three more times with water.

### Example 5

### Binding of Anticoagulant Active Ingredients to Silanized Implant Surfaces on the Example of Stent Surfaces

### 5.1 Silanizing a Stent Surface

By analogy with Example 1, the stent surface can be functionalized by a variety of aminosilanes, e.g., 3-ainopropanetrimethoxysilane or 3-aminopropanetriethoxy-silane in toluene.

The stent surfaces, in particular, in the case of stents with plastic or silicon carbide surfaces, are pretreated, if necessary, by means of conventional plasma technical methods so that hydroxyl groups on the surface, are formed and can then be coupled with ethoxy- or methoxysilanes in another step. Suitable pretreatment methods here are described, for example, in the dissertation by Alexander Borck, “Synthesis and Investigation of Biocompatible Materials for Medical Technical Applications,” University of Braunschweig; URL: http://www.digibib.tu-bs.de/?docid=00000014; chapters 2.3.2; 3.2.2.

For silanization, 100 μL triethoxypropylaminosilane is dissolved in 15 mL dry toluene. The stents are transferred to dry test tubes and overlayered with 2 mL of the silane solution. After 15 minutes, the stents are rinsed with dichloromethane and incubated for one hour at 75° C.

### 5.2 Binding the Anticoagulant Active Ingredient to the Silanized Stent Surface

Binding of the substances to stents amine-functionalized by silanization is performed like the binding in the dopaminized method.

### Example 6

### Binding of Anticoagulant Active Ingredients to Implant Surfaces Functionalized with 1,1′-carbonyldiimidazole (CDI) on the Example of Stent Surfaces

### 6.1 1,1′-Carbonyldiimidazole (CDI) Functionalization of a Stent Surface

A stent surface is brought in contact with a solution of 2.5 g 1,1′-carbonyldiimidazole (CDI) in 50 mL dioxane (anhydrous) for 5 hours at 20° C.

### 6.2 Binding of an Anticoagulant Active Ingredient to the 1,1′-Carbonyldimidazole (CDI) Functionalized Stent Surface

Coupling of one or more anticoagulant peptides is performed in a 125 mM sodium borate solution with 0.066% sodium dodecyl sulfate (SDS) at a pH of 10. The peptide concentration in this solution is 0.01-1 g peptide in 1 mL solution. The stent is immersed in the peptide solution at 5° C. for 12 hours.

### Example 7

### Simplex Gel Formation on Implant Surfaces on the Example of a Stent Surface

4 mL of a Ca alginate suspension (10%) in CaCl₂ (1%) is added to and suspended in 26 mL chitosan solution (low viscosity, 25%, Fluka). For preparation of the sodium tripolyphosphate solution, 15 g tripolyphosphate (sodium pentaphosphate, Fluka) is dissolved in 1 L double-distilled water. The pH is adjusted to 6 using 1N HCl. The pH of the alginate-chitosan suspension is adjusted at 5.5 with 1N HCl.

After adding acid, the solution is highly viscose. This solution is added by drops to 1.5% pentasodium polyphosphate solution at a pH of 6 and/or a carrier coated with the suspension is immersed in a 1.5% pentasodium polyphosphate solution at a pH of 6. After 50 minutes, the stent can be removed from the solution.

All patents, patent applications and publications referred to herein are incorporated by reference in their entirety.

What is claimed is:

1. An implant for a human or animal body, wherein the surface of the implant has a wetting angle of Θ, where Θ≦80°.

2. The implant of claim 1, wherein the implant is selected from the group consisting of cardiac pacemakers, cerebral pacemakers, cardiac implants, pacemaker electrodes, defibrillation electrodes, cochlear implants, dental implants, endoprostheses, drug depot implants, biodegradable coronary stents, permanent coronary stents, peripheral stents, biodegradable or permanent stents for other body cavities and local drug delivery (LDD) implants.

3. The implant of claim 1, wherein the implant is either a biodegradable or a permanent stent.

4. The implant of claim 3, wherein the stent base body is made of either metal or polymer.

5. The implant of claim 1, wherein the surface of the implant has a wetting angle of 0≦80°, and the implant material is a material selected from the group:
a) permanent metallic materials: 316L, nitinol and Co—Cr, where the materials may be used alone or in combination with a coating of silicon carbide (coated by the CVD process) as an implant base body, preferably as a stent base body;
b) permanent polymer base bodies: polypropylene, polyethylene, polyvinyl chloride, polymethylmethylethyl acrylate, polymethylethyl acrylate, polytetrafluoroethylene, polyvinyl alcohol, polyurethane, polybutylene terephthalate, silicone, polyphosphatene as well as their copolymers and blends or polyhydroxybutyric acid (atactic, isotactic, syndiotactic and blends thereof);
c) biodegradable metallic materials, magnesium alloys; and,
d) biodegradable polymer materials: polydioxanone; polyglycolide; polycaprolactone; polyhydroxyvaleric acid; polyhydroxybutyric acid; polylactides, preferably poly(L-lactide), poly(D-lactide), poly(D,L-lactide) and blends as well as copolymers, and poly(L-lactide-co-glycolide), poly(D,L-lactide-co-glycolide), poly(L-lactide-co-D,L-lactide), poly(L-lactide-co-trimethylene carbonate), poly-ε-capro-lactone, poly(L-lactide-co-ε-caprolactone and triblock copolymers; polyester amide; polysaccharides, chitosan, alginate, carrageenan, levan, hyaluronic acid, heparin, dextran and cellulose or cellulose derivatives, nitrocellulose, and polypeptides.

6. The implant of claim 1, wherein the surface is modified with one or more hydrophilic substances, which may be either the same or different, so that the surface of the implant has a wetting angle Θ where Θ≦80°.

7. The implant of claim 6, wherein the hydrophilic substances are selected from the group consisting of hyaluronic acid, preferably crosslinked or derivatized hyaluronic acid; chondroitin sulfate, polypeptides or oligopeptides of SEQ ID No. 1 or SEQ ID No. 2 and fragments or derivatives thereof.

8. The implant of claim 1, wherein the surface is additionally modified with at least one anticoagulant active ingredient.

9. The implant of claim 8, wherein the anticoagulant active ingredient is selected from the group consisting of anticoagulant peptides, glucosamine glycans, vitamin K antagonists, sulfated anticoagulant polymers and dendrimers.

10. The implant of claim 9, wherein the anticoagulant active ingredient is selected from the group consisting of peptides of SEQ ID No. 3 or of SEQ ID No. 4, coumarin, dicoumarol, phenprocoumon, warfarin, acenocoumarol, sulfated hyperbranched polymers, sulfated star polymers, dendrimers and sulfated dendrimers.

11. The implant of claim 1, wherein the surface further comprises a coating of at least one additional active ingredient.

12. A method for producing an implant, comprising:
a) providing an implant base body; and
b) treating the implant base body such that the surface of the implant has a wetting angle of Θ, where Θ≦80°.

13. A method of reducing the dose and/or duration of administration of a concomitant systemic medication with one or more anticoagulant active ingredients, before, during and/or after implantation in a human or animal body, comprising implanting an implant for a human or animal body, wherein the surface of the implant has a wetting angle of Θ, where Θ≦80°.

14. A method for reducing the dose or duration of administration of a concomitant systemic medication with at least one anticoagulant active ingredient, before, during or after implantation of an implant in a human or animal body, comprising implanting in a human or animal body an implant whose surface has a wetting angle of Θ, where Θ≦80°.
